(19) United States
(12) Patent Application Publication
(10) Pub. No.: US 2009/0304553 A1
(43) Pub. Date: Dec. 10, 2009

(54) TOOL AND TRAY SANITATION

(75) Inventor: Eugene I. Gordon, Mountainside, NJ (US)

Correspondence Address: DARBY & DARBY P.C., P.O. BOX 770, Church Street Station, New York, NY, 10008-0770, US

(73) Assignee: Germgard Lighting, LLC, Dover, NJ (US)

(21) Appl. No.: 12/330,452

(22) Filed: Dec. 8, 2008

Related U.S. Application Data
(63) Continuation-in-part of application No. PCT/US07/70489, filed on Jun. 6, 2007.
(60) Provisional application No. 60/811,640, filed on Jun. 6, 2006.

(51) Int. Cl. B01J 19/08; F01N 11/00; A61L 2/02; B01J 19/12

(52) U.S. Cl. 422/105; 422/292; 422/186.12; 422/186.29; 422/186; 422/186.3

(57) ABSTRACT

A system for sanitizing or sterilizing an item and storing the item in a sanitized or sterile environment. The system includes an enclosure into which a sealable package containing the item is placed. At least one ozone source is configured to introduce ozone inside the package. The ozone sterilizes the interior of the package and the item. The ozone source can include an electron beam which ionizes the oxygen, a corona discharge generated within the package, or Vacuum Ultra-Violet (“VUV”) radiation emitted into the package. A germicidal radiation source can be used to sanitize the item and generate ozone. The package containing the item is substantially transparent to germicidal radiation so that the germicidal radiation sanitizes the item as well as the interior of the package. Ultrasonic transducers can introduce ultrasonic energy into the item being sanitized, to break apart groups of clumped pathogens resident on the item.

### CLAIM OF PRIORITY

This application is a Continuation in Part of PCT Application PCT/US2007/70489 filed on Jun. 6, 2007 and published as WO 2007/146699, which claims priority pursuant to 35 U.S.C. § 119 from U.S. Provisional Patent Application Ser. No. 60/811,640 entitled “Tray and Tool Sanitizer,” filed Jun. 6, 2006, the disclosures of each of which being hereby incorporated by reference in its entirety.

### FIELD OF THE INVENTION

The present invention relates generally to the sterilization of items, and more particularly to the sterilization of surgical tools and instruments and other items using germicidal radiation or ozone within a sealed sterile container.

### BACKGROUND

In a hospital or clinic environment, the sanitation of hands and sterility of surgical or related tools used in the treatment of patients is critical to control the transmission of infectious diseases. Nosocomial infections incur a tremendous cost in terms of money and manpower required for prevention, treatment when infections occur and consequences such as illness and death. Many of these infections are the result of inadequate hand sanitation technology and practice. Furthermore, in a hospital or other medical environment it is important that surgical instruments and tools, endoscopes, their respective storage cases, trays, and medication containers, be virtually free of infectious material. Frequently, it is desirable to be able to sterilize instruments at a surgical site on-demand (i.e., immediately prior or during surgery) since they may accidentally acquire pathogens, for example, by being dropped or handled improperly. A conventional technique, autoclaving is used in an on-demand setting but it compromises the instruments or tools. Moreover, washing is ineffective since it cannot achieve pathogen inactivation or removal levels comparable with sterilization.

Sanitization is necessary as well in restaurants and school lunchrooms to provide eating utensils and trays that are sanitary. Improving the sterility or sanitary condition of the items used in these environments can significantly reduce transmission of infectious pathogens, reduce death and suffering from infections, reduce the costs associated with treating infections, and protect against establishment of strains of bacteria that resist antibiotics.

Pathogen contamination by fungus, molds, etc. limits the shelf life of various packaged foods. Elimination of these pathogens from the surface of the food and its packaging can significantly extend shelf life and have commercial importance for food manufacturers and distributors.

In small hospitals, field hospitals, surgical centers and clinics for people and animals, the standard practice with respect to surgical tools and trays is to wash and place them in boiling water or in an autoclave. Boiling water is not adequate to achieve sterilization. Properly done, autoclaving is effective in reducing residual pathogens to the requisite sterilization level. The hot, wet steam contacts all exposed surfaces. Some surfaces associated with internal volumes are not accessible to steam but then they may not be a source of infection. In any case, certain materials cannot tolerate the high temperatures associated with autoclaving (e.g., 121° C. to 134° C.). The process also may degrade the quality of an instrument, for example edge quality, and limit its useful life or require regular maintenance. Hence, the choice of materials for surgical tools and trays is limited to those that can withstand high temperature steam. Moreover, post-sterilization handling before insertion in a storage pouch can compromise sterility.

In centralized sterilization facilities fumigation with Ethylene Oxide (“ETO”), or a less flammable mixture such as ETO and carbon dioxide, is used for sanitization. After fumigation, the sterilized object is placed and sealed in a sterile pouch until it is used. However, the sterility of the item can be compromised in the process of placing the object in the pouch after sterilization. Additionally, chemical sterilization can cause damage to instruments, which is exacerbated by the long processing time. What is needed in the art is a way to sterilize the object after it is sealed in a pathogen impervious pouch a low temperature process, and a sterilization method and system that reduces the exposure time of instruments to harmful chemicals.

Post-pouch-insertion sterilization can be achieved through the use of sterilizing gas, such as ETO, which can penetrate a gas permeable, microbe impermeable material of an inner pouch sealed in a gas impermeable outer pouch into which the sterilizing gas is introduced. The sterilizing gas can reach all surfaces other than closed internal ones. This process requires many hours to complete and is thus too slow for many applications. This process is thoroughly discussed in an article by David C. Furr, entitled “Manufacturers of Sterilization Cases and Trays are Working Toward the Same Patient-Safety Goal,” published in Infection Control Today, Dec. 10, 2005, which is hereby incorporated by reference in its entirety. However, ETO, which is potentially explosive, will remain in the storage pouch thus posing a risk when opening the pouch. Additionally, ETO can leave a residue on the items in the pouch.

One further sanitization solution is the use of Gamma-Ray, X-Ray, or electron beam radiation for post-pouch-insertion sterilization. This type of radiation is used because of its reliability, safety, and cost savings over ETO fumigation. ETO has many processing variables and is toxic and expensive. The Environmental Protection Agency has recently declared ETO to be both mutagenic and carcinogenic. The residual ETO in hospital products has been reported to adversely affect hospital workers. Unlike ETO fumigation, high energy radiation sterilization imparts no toxic residuals. Benefits include the option of sterilizing certain desirable materials that could not otherwise be sterilized, sterilization of internal volumes, and using new types of packaging to better protect food products and increase shelf life. Emphasis is now being placed on cost containment for health care products so radiation is becoming quite important.

Known sterilization systems require significant investment in facilities, regulatory compliance, licensing, training of personnel, and attention since neither the ETO gas nor the radiation is safe unless properly managed. Further, ETO can not be used to sanitize foodstuffs because of residues.

What is needed in the art is a cost effective, safe, and versatile way to sterilize tools. Further, sterilization that takes place inside the final storage container or pouch without post sterilization handling is desired to decrease the exposure to pathogens and risk of contamination.

### SUMMARY OF THE INVENTION

The present invention relates to a system for sterilizing an item and storing the item in a sterile environment. The system includes a sterilizing enclosure into which a sealable package containing the item being sterilized is placed. At least one ozone source is configured to introduce ozone inside the package. The ozone sterilizes the interior volume of the package and the item contained therein.

The ozone source can include an electron beam which ionizes the oxygen contained in the package to produce ozone. Alternatively, a corona discharge can be generated within the package so as to convert the oxygen present into ozone.

In accordance with a further aspect of the present invention, a system for sanitizing an item within a sanitary package is also provided. The system includes a sanitizing enclosure having at least one germicidal radiation source configured to emit germicidal radiation. A package which is substantially transparent to germicidal radiation and contains the item being sanitized is placed within the enclosure. The germicidal radiation penetrates the package and sanitizes the item as well as the interior of the package.

In accordance with a further aspect of the present invention, the germicidal radiation source can be configured to produce radiation at a Vacuum Ultra-Violet (“VUV”) wavelength of about 185 nm or 172 nm so as to generate ozone within the pouch.

In accordance with yet a further aspect of the present invention, ultrasonic transducers can be used to introduce ultrasonic energy into the item being sanitized, thereby breaking apart groups of clumped pathogens resident on the item.

### BRIEF DESCRIPTION OF THE DRAWINGS

The foregoing and other features of the present invention will be more readily apparent from the following detailed description and drawings of the illustrative embodiments of the invention wherein like reference numbers refer to similar elements throughout the views and in which:

FIG. 1 is an illustration of a sanitizing enclosure in accordance with an embodiment of the present invention;

FIG. 2 is a further illustration of a sanitizing enclosure in accordance with an embodiment of the present invention;

FIG. 3 is an illustration of a device for sanitizing enclosed volumes in accordance with the present invention;

FIG. 4 is an illustration of a device for generating ozone in a sterilizing pouch in accordance with an embodiment of the present invention;

FIG. 5 is an illustration of a further device for generating ozone within a sterilizing pouch in accordance with an embodiment of the present invention; and

FIG. 6 is an illustration of a further device for generating ozone within a sterilizing pouch in accordance with an embodiment of the present invention.

### DESCRIPTION OF CERTAIN EMBODIMENTS OF THE INVENTION

It is possible to sanitize by deactivation of surface pathogens using germicidal radiation, such as UVC radiation, typically produced at λ253.7 nm continuously from an argon-mercury gas discharge lamp. Sanitation can also be achieved using UVC radiation from a pulsed xenon arc and other known methods.

The use of ETO gas and/or gamma ray irradiation for purposes of achieving sterility of in-pouch tools, trays, and other devices is generally known in the art. However, ETO gas and gamma-ray irradiation are inconvenient and expensive. The present invention utilizes ozone in combination with, and within a sealed storage container (e.g., a pouch), to effectively and cost-efficiently sterilize.

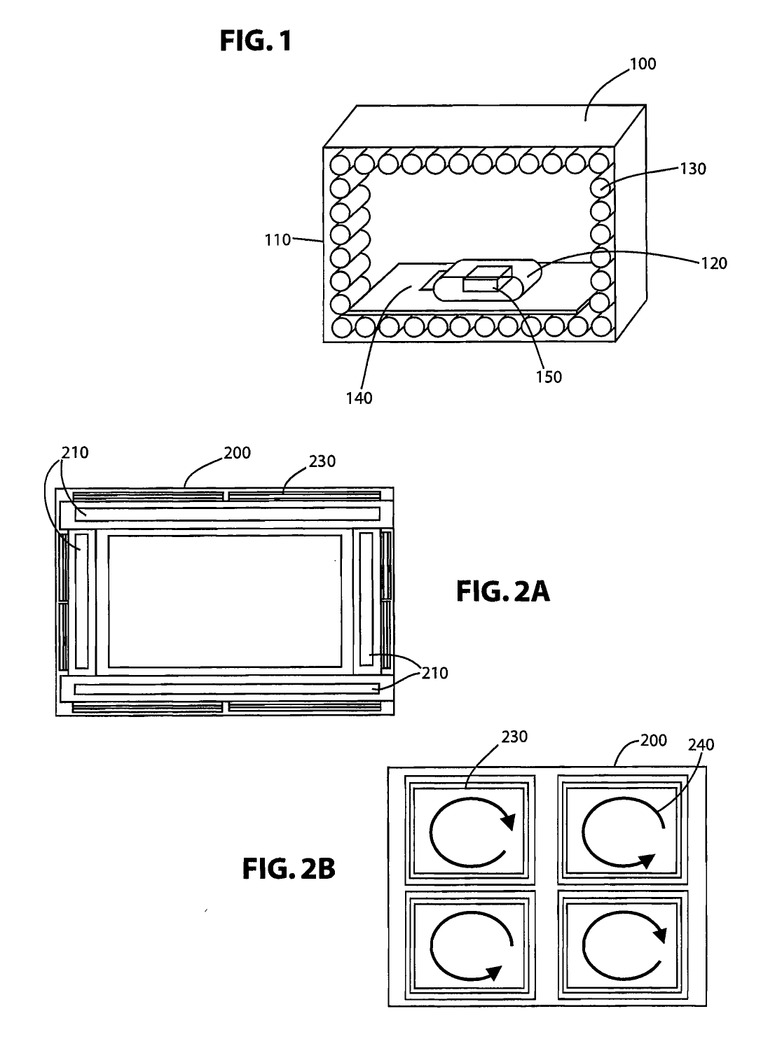

FIG. 1 illustrates a sterilizing box 100 into which the items 150 to be sanitized by UVC are placed. Inside the box 100 is an array of linear germicidal bulbs 130 that produce a high internal flux of UVC (i.e., radiation having a wavelength of 253.7 nm). The inner surface 110 of the box walls is preferably reflecting to maintain the highest UVC flux level within the box. Within the volume of the box 100 the UVC flux is isotropic and has a substantially uniform intensity under normal conditions of operation. Hence, the intensity everywhere in the box can be maintained as a sufficient intensity.

Higher intensities of UVC radiation allow shorter sanitation times and greater percentage of pathogen deactivation (i.e., inactivation). If the UVC emitted from the opposite side of the germicidal bulbs 130 is reflected (e.g., by UVC reflecting inside walls 110), then even greater intensities can be achieved. Aluminum, for example, provides an excellent UVC reflector. Further, the lifetime of conventional A/C germicidal tubes can be compromised in the interest of operating at higher intensity.

The intensity of the UVC radiation can also be increased by using planar RF-excited discharge devices, rather than conventional germicidal bulbs 130. FIG. 2 illustrates a possible placement of planar RF-excited discharge devices 210 within a sanitization box 200. Discharge devices 210 can be placed on all inner walls including the door. The direction of the current flow in each coil 230 is chosen to maximum the induced electrical field to produce the maximum discharge intensity. The arrows 240 illustrate the directions of current flow in the coils 230 to maximize the induced fields in the central regions.

The box 100 preferably has a door and the closed structure serves to contain UVC, and RF if RF-excited tubes are used. A platform 140 preferably made of a low UVC transmission loss material (e.g., quartz) can be included in the box 100 to support items 150 to be sanitized. The quartz platform minimally attenuates the UVC radiation.

As an example consider that one of the most difficult pathogens to deactivate is the anthrax spore. The anthrax spore requires a dose of about 700 Joules/meter² of radiation at λ253.7 nm for 90% deactivation. Using standard lamps producing 225 watts/meter² of such radiation at the tube surface, an anthrax spore in the immediate vicinity of the tube envelope requires approximately 3.1 seconds for 90% deactivation; approximately 1400 Joules/meter² for 99% (6.2 seconds); and approximately 2800 Joules/meter² for 99.99% deactivation (12.5 seconds). Because the intensity falls off with distance from a line source, the required exposure time increases proportionally to distance from the tube. An exposure time of approximately 12.5 seconds can be just adequate to achieve 99.99% deactivation for an object immediately adjacent to the tube surface but not all parts of an object may be so close. These exposure times can be reduced by positioning the tubes and using reflective walls to increase the useful intensity and make the radiation distribution substantially uniform and isotropic. Alternatively, lamps with much higher output radiation levels, or pulsed output, can achieve shorter exposure times.

The item 150 being sanitized is preferably inserted into a pouch 120 and sealed prior to being inserted into the box 100. The pouch is preferably made of a film that is relatively transparent to UVC radiation such as Acrylite OP-4. Such a pouch would serve as a sanitization pouch as well as a storage pouch. Thus, once the item in the pouch is sanitized, along with the item, it can remain inside the pouch without further handling. It is not necessary to transfer the item to an additional storage container, during which process the item would be subject to potential contamination.

With respect to OP-4, the transmission factor for the single surface UVC reflection loss of the pouch can be approximated as T_{R}=0.96. For the transmission loss of the bulk material, T_{L},

T_{L}=exp−α

[symbol]

in which α is the absorption factor and [symbol] is the thickness. Hence, the overall transmission factor, T, is:

T=T_{R}²T_{L}=0.92 exp−α

[symbol]

T_{R} is the reflection factor of a single surface, typically about 0.96. Thus, a rough estimation of T for a material having a thickness of 0.100 inches is T≈0.15 for λ254 nm radiation. Hence, T_{L}≈0.16, α [symbol] ≈1.8 and since [symbol] =100 mils, α≈1.8×10⁻² mils⁻¹. This allows construction of the following table for OP-4 film loss.

The following table shows that, for thin sheets for which the transmission loss due to absorption is low, T_{L}≈1−α

[symbol]

| [symbol] (mils) | T_{L} | T |
|---|---|---|
| 1 | 0.982 | 0.90 |
| 2 | 0.965 | 0.89 |
| 3 | 0.947 | 0.87 |
| 10 | 0.835 | 0.77 |

Alternatively, the pouch 120 can be made from a thin film of fused quartz. Certain types of quartz are highly transparent to UVC radiation and would increase the efficiency of the sanitization system. However, high UVC transmission quartz can be very costly. Thus, the pouch 120 can include quartz that has been pulled into a fiber and woven into a fabric. Such a quartz woven pouch 120 provides physical strength and is highly transparent to UVC at the preferred wavelengths. The woven quartz pouch 120 can be coated with a thin layer to ensure that it is relatively gas and pathogen impermeable.

A suitable thickness for the quartz pouch can be in the range 25-50 microns (i.e., 0.025-0.050 mm) so the absorption loss at λ254 nm is negligible. Higher quality quartz can be used to produce a pouch suitable for transmitting VUV at λ185 nm. To produce pouch material, quartz rods can be heated to the softening temperature and rolled into sheet. Heating and fusing the edge seals the pouch. The quartz sheet is strong enough and flexible enough to create an operable pouch. Alternately, quartz rods can be pulled into a fiber about 50 microns in diameter, similar in physical structure to low loss optical transmission fiber. The fiber can then be woven into cloth. The quartz cloth is transparent at λ185 nm, however will not likely block transmission of ozone through its interstices. Thus, the quartz cloth is preferably coated with a thin layer of resin or equivalent such that diffusion of ozone is blocked. Because the coating is only microns thick the coating has low transmission loss for λ185 nm.

While quartz and OP-4 have been discussed as a suitable pouch material, any material that is not highly absorbent at λ185 nm can be used to create the pouch. Further, since the pouch material can be extremely thin, the range of suitable materials is broadened due to the minimal transmission loss of the VUV radiation through the thin pouch material.

When a pouch is used to enclose the item(s) being sanitized, the necessary exposure time can be increased by a factor equal to the reciprocal of the pouch material transmission or absorption loss factor. Since transmission factors can be in the range of 0.80 to 0.90, the necessary exposure times can be increased by about 10-20%. As noted, shorter exposure times, and better levels of deactivation are possible using higher intensity lamps, better lamp placement, and means to eliminate pathogen clumping.

UVC and other sanitization methods only sanitize surfaces and deactivate those pathogens with which it comes in contact. Thus, any pathogen clumping can decrease the effectiveness of any sanitation method. To increase the level of surface exposure and pathogen contact, an ultrasonic transducer, preferably operating at a frequency of about 40 KHz, can be coupled to a UVC transmissive plate 140 (e.g., quartz plate) and can introduce ultrasonic energy to the item to be sterilized. This ultrasonic energy breaks up the clumped pathogens so that individual pathogens are exposed to the UVC.

It should be noted that UVC does not reach some internal surfaces because of shadowing. Many items, such as instrument trays and cases, have only open surfaces and do not present a problem. Closed volumes are not a significant problem since pathogens and contaminants generally can not easily escape the closed volume. However, many instruments can include small openings to the outside environment through which pathogens can enter, resulting in contamination of the inner volume. In part, because of shadowing, UVC flux does not reach all surfaces of the inner volumes. The effects of shadowing can be overcome through various solutions.

Ozone is an alternative, or complementary, approach to sterilizing internal volumes that have an opening. Ozone can achieve a deactivation level of 10⁻⁶, by rapidly oxidizing organic substances with which it comes in contact. Thus, exposing pathogens on the surface or internal volume of an item to a sufficient concentration of ozone can kill the pathogens and prions, and thus disinfect the item. Ozone production can be achieved with Vacuum Ultra-Violet (“VUV”) lamps, preferably designed to produce λ185 nm radiation. Such lamps are commercially available for producing ozone. Other techniques for producing ozone can be used, as described below. Thus, in accordance with a further feature of the present invention, the germicidal bulbs 130 produce radiation having a VUV wavelength, preferably of about 185 nm. Radiation at this wavelength converts the oxygen within the pouch 120 to ozone. The ozone permeates the enclosure and disinfects all surfaces with which it comes in contact including inner volumes or shadowed spaces

When oxygen converts to ozone, the total number of molecules, or moles, reduces, thus reducing the internal volume of the pouch at atmospheric. The compression of the pouch provides a qualitative, visual indication that ozone has formed.

Ozone is more effective in a high humidity ambient since it interacts with water or water vapor to produce OH⁻, which is highly destructive of pathogens. Thus, optionally, water vapor can be introduced to the box 100 to increase the production of OH⁻. However, the instrument would not be dry when ready for use.

Ozone is potentially harmful to humans. However, there are many simple ways to eliminate ozone before the pouch 120 or box 100 is opened. For example, the ambient or local temperature of the box 100 and the passage of time at high temperature eliminate ozone. The following table shows the half-life of ozone at varying temperatures, when surfaces play no role. Surfaces can shorten these times.

Typical O₃ half-life vs. Temperature

| Temp (C.) | Half-life* |
|---|---|
| −50 | 3-months |
| −35 | 18-days |
| −25 | 8-days |
| 20 | 3-days |
| 120 | 1.5-hours |
| 250 | 1.5- seconds |

Alternatively, the ozone generated in the enclosure can be exhausted through a heated, sintered element such as charcoal or hot stainless steel, which converts ozone to oxygen on contact. A small tube heated to a temperature above 250° C. is one example of an ozone deactivation device. Optionally, a combination of ozone deactivation techniques can be combined.

The box 100 can further include a safety mechanism on the door to prevent the box 100 from being opened prior to deactivation of ozone to safe levels. The level of ozone can be measured by sensors in the box 100. However, because unforeseen circumstances may arise in which it is necessary to access the inside of the box regardless of any remaining ozone, an override mechanism can be included to allow a user to disengage the safety mechanism preventing the box from being opened, thereby allowing the door to be opened.

Alternatively, a pouch used in the disinfecting process can be used to localize the ozone to the volume within the pouch, thus eliminating many of the risks associated with human exposure to ozone when opening the box to remove the pouch. Additionally, because the pouch comprises a sealed environment, once the sterilization process is completed, the pouch maintains the sterile environment in which the items reside, until the pouch is opened.

Therefore, once the pouch 120 is inserted in the box 100, the box interior can be flushed with nitrogen so as to replace the air in the box. Alternatively, the box can be pumped down to eliminate most of the internal air which is then filled with nitrogen. Thus, when the VUV is introduced into the enclosure 100, insufficient oxygen will be present between the enclosure walls and the pouch to generate ozone within this volume.

It should be noted that if the production of ozone is undesirable, the wall of the germicidal source (i.e., the wall of the tube) can be made from a material that absorbs VUV radiation, such as Quartz-L.

In accordance with yet a further feature of the present invention, ozone can be introduced into the pouch 120 by exhausting the closed pouch, producing a weak vacuum, and then introduce ozone made externally through a valve, for example through a Schraeder valve that is incorporated into the pouch envelope. The pouch is filled with ozone to a pressure approximating atmospheric pressure. As the ozone in the pouch decays to oxygen, two ozone molecules become three oxygen molecules, increasing the associated molar content by 50% and the pouch expands slightly, thus providing a visual indication that the ozone is no longer present and it is safe to open the pouch without emission of ozone into the atmosphere.

Alternatively, the package can be filled with O₂, which is subsequently converted to ozone. The conversion to ozone can be achieved through a coronal discharge, as described herein, or other another known means of generating ozone. It is preferable to create the ozone within the sealed package, to prevent recontamination when the item is moved from the sanitizing package to the storage package. That is, using the sterilization package as the storage package reduces the risk of recontamination.

One preferable way of creating ozone within the package includes a thin VUV transparent window, such as quartz, on the surface of the package. The thin window allows VUV to enter the internal space and produce ozone in situ. Absorption of VUV causes the O₂ molecule to disassociate into two oxygen atoms, and each oxygen atom quickly combines with an O₂ molecule to produce an O₃ molecule. Atomic oxygen is highly reactive but fortunately does not remain in its atomic stat for long in the presence of O₂.

VUV at a wavelength of 185 nm has negligible ability to inactivate pathogens directly. Some VUV producing lamps can also produce UVC at λ253.7 nm, which can inactivate pathogens directly on any surfaces that can be illuminated. While ozone can destroyed by UVB radiation, the typical germicidal lamp does not produce UVB.

A further alternative using high energy ionization of oxygen to produce ozone is illustrated by the system 600 in FIG. 6. In system 600, the electrons are created, accelerated and emitted at high kinetic energy from the cathode tube 610 in the range 10 to 20 KeV. The electrons pass through an opening 620 in the anode 630 and through the transmission window 640 into the sterilization space of the package 650. The transmission window 640 and the anode 630 can be physically separate components or attached.

The transmission window 640 is typically made of ceramic foil or other material substantially permeable to electrons of 10-20 KeV kinetic energy. Preferably, the window 640 is made of carbides, nitrides, hydrides and oxides of metals such as crystal silicon, poly-silicon, aluminum, and boron. Combinations of these materials may also be employed. The foil is preferably about 100 to about 300 nm thick, and is substantially transparent to electrons in the low-energy range discussed above.

The window 640 does not absorb more than about 5% of the kinetic energy of a 20 KeV electron passing through it. As discussed below, the window material is preferably conducting or coated with a conducting layer.

The electron beam originates from a heated or cold cathode 610 held at a voltage below ground potential. The anode 630, the window 640 and the package 650 are preferably substantially at ground potential. Electrons injected into the sterilization volume of the package 650 can be returned to the cathode 610 through the external circuit 660 associated with the window 640. The beam device can be powered by a DC voltage or an AC voltage from a high voltage transformer.

The transmission window 640 can be coupled to the package 650 through known methods. For example, the beam device can be coupled by a threaded fitting and include an O-ring seal to prevent leaking from the package 650. A slide fit between the device and the package 650 can also be used.

Electrons are thus injected through the foil window 640 directly into the oxygen gas. A net negative charge can collect at the output side of the window 640 and repels new electrons entering the space. These electrons can be drained off to avoid interference with the beam penetration of the window 640. Thus, the exterior side of the foil window 640 can be coated with a conductor such as a thin layer of metal, or the material used for the foil window 640 can be conductive. Application of a small positive voltage to the conducting window 640 can be used to drain off the space charge and prevents beam blockage. The high energy electrons impacting on oxygen create ozone by processes similar to those occurring in a corona discharge.

Ozone can also be generated within the pouch by system 400 illustrated in FIG. 4. In this system 400, an A/C current source 430 connected to a primary coil 440 can induce a high voltage in a secondary coil 450 located inside the pouch 420 filled with oxygen or air 410. The A/C source can be operating at a frequency as low as 60 Hz but is preferably at around 250 kHz. The secondary coil 450 preferably has a high turns ratio so as to induce a high voltage. The voltage across spark gap 460 within the pouch induces a gas discharge breakdown across the spark gap 460. The resistor 470 connected to the primary coil and the coil inductance limit the gap current so the discharge is controlled. The spark gap 460 discharge converts oxygen or air 410 within the pouch 420 to ozone. The primary coil 440 on the external side of the pouch 420 can be part of the pouch 420 assembly and processing equipment. The secondary coil 450 and spark gap 460 inside the pouch 420 could be made very inexpensively as thin film elements on a support structure such as glass or plastic so as to be disposable.

The concentration of ozone generated depends on the percentage of oxygen in the original gas fill, the discharge current, and the operation time. The lifetime of the ozone in the pouch could be days or minutes depending on the ambient temperature and the volume. As previously discussed, if necessary, the pouch could be heated just prior to opening to eliminate the residual ozone.

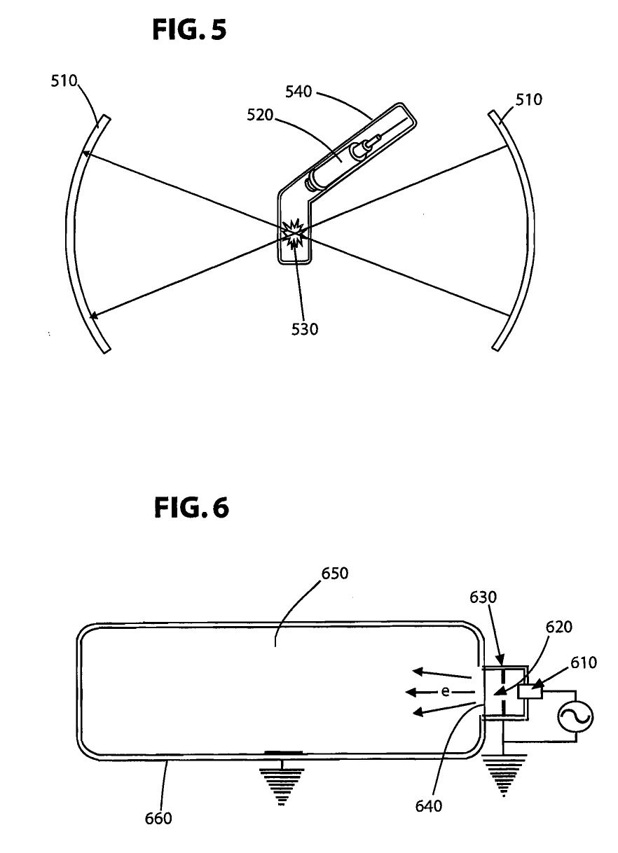

In yet a further ozone production alternative, a microwave cavity arrangement 500, as illustrated in FIG. 5, including two antennas 510 with a spherical focusing surface is provided so that short wavelength microwaves are focused to a small volume 530. A characteristic dimension of the focused volume 530 is roughly the microwave wavelength, and the small volume 530 is preferably the region of highest microwave intensity. The system has many resonant wavelengths, and the energy density within the focus region can be quite high. Adequate power at a resonant frequency applied to the air or oxygen in the vicinity of the focused spot causes the oxygen to break down and a small, high intensity gas discharge plasma region develops. In this region, oxygen is converted to ozone under the action of the plasma and the associated optical radiation. A pouch 520 with an instrument 540 to be sterilized can be placed so that the breakdown region is within the pouch 520. The breakdown produces ozone, which fills the pouch 520 and sterilizes the included instrument 540.

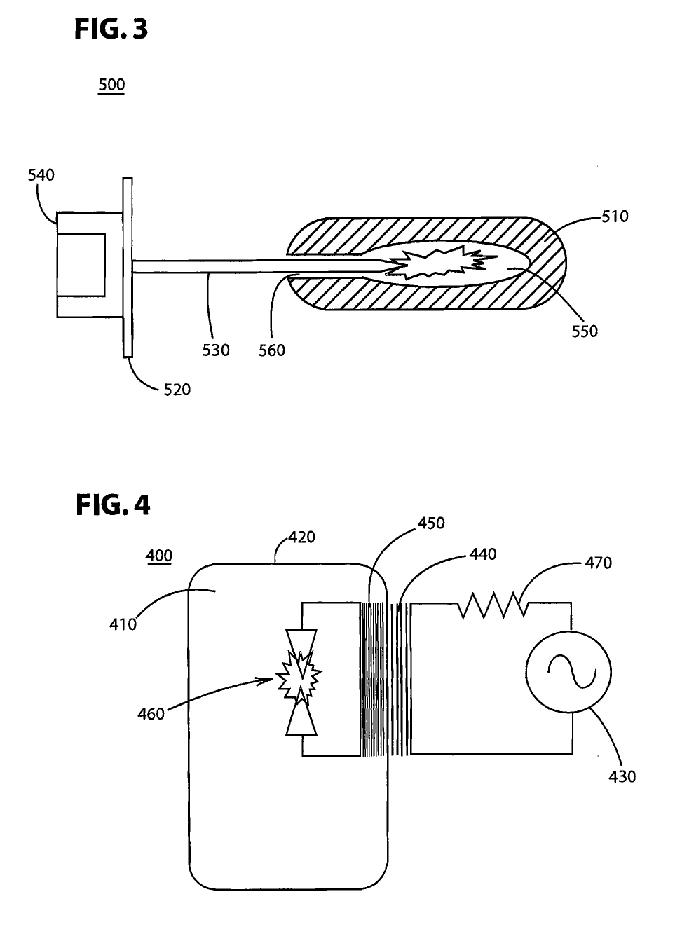

In accordance with yet a further feature of the present invention, the use of ozone to sanitize internal volumes can be avoided or complemented by the device illustrated in FIG. 3. FIG. 3 illustrates item 310 with an internal volume 350 having an opening 360. The opening 360 is not large enough to allow significant UVC flux to penetrate to the internal volume 350. However, substantial UVC flux can be guided into the volume using a quartz rod 330 coupled to a wall of the discharge tube 340 through the wall of the pouch 320. Alternatively, small, high intensity lamps can be used to bring high flux levels into a guiding rod through the wall of the pouch 320 and into the internal volume 350. This flux permeates the internal volume 350 and sanitizes the associated surfaces.

In another embodiment a VUV tube can be placed inside the pouch and excited externally. The VUV radiation at λ185 nm or λ172 nm directly produces ozone inside the pouch.

The above described aspects of the invention effectively sanitize items within a pouch within a reasonable time period. However, this time period can be further reduced by combing any of the above with a pre-processing step.

Spores, by virtue of their protective shell, are relatively difficult to inactivate by exposure to ozone and require a substantial increase in the process time. However, spores placed in water at around 100 C will quickly vegetate and then can be inactivated in times that are typical of other non spore bacteria inactivation. Thus, boiling anthrax spores in water for approximately 5 to 10 minutes causes those spores to vegetate and then be inactivated by exposure to ozone to the order of 10⁻⁶ or better. Other spore types are also inactivated by short exposure to boiling water. Therefore, after the standard cleaning of the instrument, a 5 minute presoak in boiling water to vegetate various spores, followed by exposure to sterilizing ozone gas, would quickly inactivate spores as well as bacteria and virus. Furthermore, applying a rough vacuum to the surface above the boiling water ensures that any inner volumes of the instruments are filled with the boiling water so that spores within such volumes are also vegetated.

The exposure of the spore to boiling water weakens the tough outer shell of a pore and vegetates the spore. A vegetated spore can be quickly destroyed by ozone, similar to non-spore bacteria. Thus, a presoak in water at 100 C for five minutes to inactivate spores followed by an exposure to ozone gas sterilization process for a few minutes reduces the overall process time for ozone sterilization time to of order 10 minutes or less. Other chemical sterilization processes such as ethylene oxide or VH₂O₂ would also be shortened considerably.

After removal from the presoak the instrument can be placed in a sterile pouch for processing or it can be placed in the open sterile pouch before presoaking and then introduced to the gas process. While the invention has been described in connection with a certain embodiment thereof, the invention is not limited to the described embodiments but rather is more broadly defined by the recitations in the claims below and equivalents thereof.

I claim:

1. A system for sterilizing an item and storing the item in a sterile environment, the system comprising:
a package having an interior volume for containing the item being sterilized, the package being sealable and substantially impermeable to ozone; and
at least one ozone source configured to introduce ozone inside the package;
wherein the ozone sterilizes the interior volume of the package and the item contained therein.

2. The system of claim 1, wherein the ozone source includes:
a current source;
a primary coil connected to the current source and located outside of the package; and
the package includes:
a secondary coil located within the package and configured so as to derive a voltage from the primary coil; and
a spark gap connected to the secondary coil such that the voltage across the spark gap and an associated corona discharge generate ozone within the package.

3. The system of claim 1, further comprising at least one microwave source having a focusing surface focused so as to generate a high microwave intensity within the package,
wherein the package has at least one dimension that is approximately a multiple of a resonant frequency of the microwave, and
the microwave power focused at within the package results in a gas discharge plasma thereby generating ozone within the package.

4. The system of claim 1, further comprising:
a sterilizing enclosure; and
at least one radiation source configured to emit radiation having a predetermined wavelength within the sanitizing enclosure,
wherein at least a portion of the package is substantially transparent to the emitted wavelength of radiation, so as to expose the item to the radiation.

5. The system of claim 4, wherein the sanitizing enclosure includes at least one interior wall that reflects the predetermined wavelength of radiation.

6. The system of claim 4, wherein the predetermined wavelength includes VUV radiation, and the VUV radiation entering the package generates ozone in the interior volume of the package.

7. The system of claim 6, wherein the predetermined wavelength of the at least one radiation source includes at least one of about 185 nm and about or 172 nm.

8. The system of claim 4, wherein the at least one radiation source is coupled to the package proximate the portion of the package substantially transparent to the emitted radiation.

9. The system of claim 1, wherein the package includes a transmission window substantially permeable to electrons in a predetermine kinetic energy range, the system further comprising an electron beam source configured to emit an electron beam toward the transmission window so as to ionize gas contained in the package and generating ozone within the package.

10. The system of claim 9, wherein the transmission window comprises a ceramic foil.

11. The system of claim 9, wherein the electron beam source includes a cathode and an anode.

12. The system of claim 9, wherein the electron beam source is coupleable to the transmission window.

13. The system of claim 1, further comprising:
a sterilizing enclosure;
an ozone sensor configured to measure the ozone within the enclosure; and
a lockable opening coupled to the enclosure, the lockable opening configured to lock while the ozone sensor senses a predetermined concentration of ozone in the enclosure.

14. The system of claim 1 further comprising:
an ultrasonic transducer coupled to the package so as to introducing ultrasonic energy to the package and the item contained therein and declump pathogens.

15. The system of claim 1, further comprising:
a sterilizing enclosure; and
an ozone removal device coupled to the enclosure.

16. The system of claim 15, wherein the ozone removal device includes a heated element through which the gas inside the enclosure is exhausted.

17. The system of claim 1, further comprising a hot water bath configured to receive the item being sterilized for a predetermined period of time, prior to insertion of the item into the package.

18. The system of claim 17, wherein the hot water bath can boil water placed within the bath.

19. The system of claim 17, wherein the hot water bath is further configured to produce a vacuum above the surface of the water.

20. A system for sanitizing an item within a sanitary package, the system comprising:
a sanitizing enclosure;
at least one germicidal radiation source configured to emit germicidal radiation within the sanitizing enclosure;
a package for containing the item and being placed within the sanitizing enclosure, the package comprising a material that is substantially transparent to germicidal radiation.

21. The system of claim 20, wherein the sanitizing enclosure includes at least one interior wall that reflects germicidal radiation.

22. The system of claim 20 further comprising an ultrasonic transducer coupled to the package so as to introducing ultrasonic energy to the package and the item contained therein and declump pathogens.

23. The system of claim 20, wherein the at least one germicidal radiation source substantially lines the interior of the sanitizing enclosure.

24. The system of claim 20, wherein the package comprises woven quartz.

25. The system of claim 20, wherein the package comprises OP-4.

26. The system of claim 20, wherein the at least one germicidal radiation sources comprises a planar RF-excited radiation source.

27. The system of claim 20, wherein the package further comprises a protrusion for insertion into shadowed volumes of the item, the rod configured to introduce germicidal radiation within the volume.

28. The system of claim 27, wherein the protrusion includes a quartz waveguide, the protrusion being coupleable to the germicidal radiation source.

29. The system of claim 27, wherein the protrusion includes a second germicidal radiation source at a distal end of the protrusion.

30. The system of claim 20, wherein the germicidal source produces VUV radiation so as to generate ozone within the pouch.

31. The system of claim 20, further comprising:
a current source;
a primary coil connected to the current source and located outside of the package;
a secondary coil located within the package and configured so as to induct a voltage from the primary coil; and
a spark gap connected to the secondary coil such that the voltage across the spark gap generates ozone within the package.

32. The system of claim 20, further comprising a hot water bath configured to receive the item being sterilized for a predetermined period of time, prior to insertion of the item into the package.

33. The system of claim 32, wherein the hot water bath can boil water placed within the bath.

34. The system of claim 32, wherein the hot water bath is further configured to produce a vacuum above the surface of the water.
